(19) United States
(12) Patent Application Publication
(10) Pub. No.: US 2026/0290514 A1
(43) Pub. Date: Sep. 24, 2026

(54) METHOD AND APPARATUS FOR MOLECULAR DYNAMICS SIMULATION

(71) Applicant: SAMSUNG ELECTRONICS CO., LTD., Suwon-si (KR)

(72) Inventors: Ying ZHANG, Xi’an (CN); Wenlong HE, Xi’an (CN); Yujie ZENG, Xi’an (CN); Vasyltsov IHOR, Suwon-si (KR); Zihao LIU, Xi’an (CN); Jiaxin WEI, Xi’an (CN); Lin CHEN, Xi’an (CN); Yuehua DAI, Xi’an (CN)

(21) Appl. No.: 19/539,530

(22) Filed: Feb. 13, 2026

(30) Foreign Application Priority Data
Mar. 21, 2025 (CN) 202510344192.6
Oct. 24, 2025 (KR) 10-2025-0155676

(51) Int. Cl. G16C 10/00; G06F 16/245; G06F 30/20

(52) U.S. Cl. CPC G16C 10/00; G06F 16/245; G06F 30/20

(57) ABSTRACT

A method includes determining, using a machine-learning model, an interaction force between interacting atoms of a preprocessed atomic graph including an original atomic graph and a mirrored replicated atomic graph surrounding the original atomic graph, wherein the mirrored replicated atomic graph is generated by performing spatial decomposition of a simulation space in the original atomic graph, adjusting magnitudes of two forces of each pair of pairwise interaction forces of the interacting atoms, determining a net force applied to each of the interacting atoms in the original atomic graph based on the adjusted magnitudes, and generating an updated the position of each atom based on the net force.

### CROSS-REFERENCE TO RELATED APPLICATIONS

This non-provisional patent application claims priority under 35 USC § 119 to Chinese Patent Application No. 202510344192.6, filed on Mar. 21, 2025, in the China National Intellectual Property Administration, and Korean Patent Application No. 10-2025-0155676, filed on Oct. 24, 2025, in the Korean Intellectual Property Office, the entire disclosures of which are incorporated herein by reference.

### TECHNICAL FIELD

Embodiments of the present disclosure relate to the field of molecular dynamics learning, and more particularly, to a method and apparatus for molecular dynamics simulation.

### DISCUSSION OF RELATED ART

Molecular dynamics (MD) is a method of simulating materials at an atomic scale by solving equations of motion of atoms based on Newtonian mechanics to numerically model interactions among atoms, molecules, or particles. MD enables understanding of dynamic characteristics of materials and biological systems by observing changes of a simulated system state over a plurality of discrete simulation time steps. Such dynamic characteristics may provide improvements in various industrial application fields, including material design, catalyst research, drug development, protein structure analysis, and other applications that may need the analysis of time-evolving atomic-scale behavior.

Since a material (e.g., a metal, compound, etc.) to be simulated may include a large number of atoms, such as millions to billions of atoms, dividing a simulation space into a plurality of subspaces and performing an MD simulation in parallel across a plurality of processing elements is an effective solutions for performing large-scale MD simulations.

Conventional MD simulation methods may be classified into the following three techniques. First is a classical approximation methods, such as methods based on Lennard-Jones potentials and Tersoff potentials, provide fast simulation speed and excellent scalability, but may exhibit low accuracy. Second is first-principles calculation methods based on density functional theory enable accurate simulations, but are computationally expensive and limited to simulations involving several tens to at most several hundreds of atoms. Third is machine-learning-based methods mainly use two types of force prediction approaches, including a gradient-force approach and a direct-force approach, which each involve a trade-off between speed and accuracy. For example, the gradient-force approach provides high accuracy but exhibits slow computational speed, while the direct-force approach provides fast computational speed but exhibit low accuracy.

### SUMMARY

A method and an apparatus for molecular dynamics simulation, which increase simulation speed while improving accuracy of molecular dynamics simulation are provided.

In one aspect, an electronic device includes at least one processor and a memory storing instructions, in which the instructions, when executed individually or collectively by the at least one processor, cause the electronic device to determine, using a machine-learning model, an interaction force between interacting atoms of a preprocessed atomic graph including an original atomic graph and a mirrored replicated atomic graph surrounding the original atomic graph, the mirrored replicated atomic graph is generated by performing spatial decomposition of a simulation space in the original atomic graph, adjust magnitudes of two forces of each pair of pairwise interaction forces of the interacting atoms, determine a net force applied to each of the interacting atoms in the original atomic graph based on the adjusted magnitudes, and predict the position of each atom based on the net force.

The pairwise interaction forces may include pairwise interaction forces obtained in the same simulation process and pairwise interaction forces obtained in different simulation processes, in which among the pairwise interaction forces obtained in the same simulation process, forces may be obtained in the same simulation process, and among the pairwise interaction forces obtained in the different simulation processes, a first force may be obtained by a first simulation process and a second force may be obtained by a second simulation process different from the first simulation process.

The instructions, when executed individually or collectively by the at least one processor, may cause the electronic device to obtain a first preprocessed atomic graph and a second preprocessed atomic graph by spatially dividing the preprocessed atomic graph, determine forces between interacting atoms of the first preprocessed atomic graph through the first simulation process, and determine forces between interacting atoms of the second preprocessed atomic graph through the second simulation process, in which the first simulation process and the second simulation process may be executed as parallel simulation processes.

The instructions, when executed individually or collectively by the at least one processor, may cause the electronic device to assign an identifier to each atom of an original atomic graph, assign the identifier to each atom in the mirrored replicated atomic graph corresponding to each atom of the original atomic graph, and match pairwise interaction forces based on the identifier of atoms of the preprocessed atomic graph.

The instructions, when executed individually or collectively by the at least one processor, may cause the electronic device to synchronize the matched pairwise interaction forces through inter-process communication based on the Message Passing Interface (MPI) between the first simulation process and the second simulation process.

The instructions, when executed individually or collectively by the at least one processor, may cause the electronic device to align pairwise edges corresponding to the pairwise interaction forces based on the identifier assigned to atoms associated with the pairwise edges, and synchronize the matched pairwise interaction forces between the first simulation process and the second simulation process based on the aligned pairwise edges.

The instructions, when executed individually or collectively by the at least one processor, may cause the electronic device to map the preprocessed atomic graph to a lambda space including a plurality of subspaces, determine an identifier of a subspace corresponding to a first atom of two atoms of each interaction force pair, determine a second identifier of a subspace corresponding to a second atom of the two atoms, and determine whether to adjust each interaction force pair based on a comparison of the first identifier and the second identifier.

The instructions, when executed individually or collectively by the at least one processor, may cause the electronic device to compute an average value of magnitudes of two forces of pairwise interaction forces.

The machine-learning model may include a graph neural network and a plurality of force prediction networks, the graph neural network may include a plurality of cascaded layers, and the instructions, when executed individually or collectively by the at least one processor, may cause the electronic device to input the preprocessed atomic graph to a first layer of the graph neural network to generate an output feature, and determine forces between interacting atoms of the preprocessed atomic graph based on the output feature.

In another aspect, a method performed by an electronic device includes determining, using a machine-learning model, an interaction force between interacting atoms of a preprocessed atomic graph including an original atomic graph and a mirrored replicated atomic graph surrounding the original atomic graph, wherein the mirrored replicated atomic graph is generated by performing spatial decomposition of a simulation space in the original atomic graph, adjusting magnitudes of two forces of each pair of pairwise interaction forces of the interacting atoms, determining a net force applied to each of the interacting atoms in the original atomic graph based on the adjusted magnitudes, and generating an updated the position of each atom based on the net force.

The pairwise interaction forces may include at least one of pairwise interaction forces obtained in the same simulation process and pairwise interaction forces obtained in a different simulation process, among the pairwise interaction forces obtained in the same simulation process, forces may be obtained in the same simulation process, and among the pairwise interaction forces obtained in the different simulation processes, a first force may be obtained by a first simulation process and a second force may be obtained by a second simulation process different from the first simulation process.

The determining of the force between the interacting atoms of the preprocessed atomic graph may include obtaining a first preprocessed atomic graph and a second preprocessed atomic graph by spatially dividing the preprocessed atomic graph, determining forces between interacting atoms of the first preprocessed atomic graph through the first simulation process, and determining forces between interacting atoms of the second preprocessed atomic graph through the second simulation process, in which the first simulation process and the second simulation process may be executed as parallel simulation processes.

The method may further include assigning an identifier to each atom of an original atomic graph, assign the identifier to each atom in the mirrored replicated atomic graph corresponding to each atom of the original atomic graph, and matching pairwise interaction forces based on the identifier of atoms in the preprocessed atomic graph.

The method may further include synchronizing the matched pairwise interaction forces between the first simulation process and the second simulation process based on the MPI.

The synchronizing of the matched pairwise interaction forces may include aligning pairwise edges corresponding to the pairwise interaction forces based on the identifier assigned to atoms associated with the pairwise edges and synchronizing the matched pairwise interaction forces between the first simulation process and the second simulation process based on the aligned pairwise edges.

The method may further include mapping the preprocessed atomic graph to a lambda space including a plurality of subspaces, determining a first identifier of a subspace corresponding to a first atom of two atoms corresponding to each interaction force pair, determining a second identifier of a subspace corresponding to the other atom of the two atoms based on the first identifier, and determining whether to adjust each interaction force pair based on a comparison of the first identifier and the second identifier.

The adjusting of the two forces may include computing an average value of magnitudes of two forces of pairwise interaction forces.

The machine-learning model may include a graph neural network and a plurality of force prediction networks, the graph neural network may include a plurality of cascaded layers, and the determining of the force between the interacting atoms of the preprocessed atomic graph may include inputting the preprocessed atomic graph to a first layer of the graph neural network to generate an output feature, and determining forces between the interacting atoms of the preprocessed atomic graph based on the output feature.

### BRIEF DESCRIPTION OF THE DRAWINGS

FIG. 1 illustrates an example comparing conventional generation of ghost atoms by replicating local atoms and generation of ghost atoms by replicating local atoms according to an embodiment of the present disclosure.

FIG. 2 illustrates an example of predicting pairwise forces.

FIG. 3 illustrates a flowchart of a molecular dynamics (MD) simulation method according to an embodiment.

FIG. 4 illustrates an example of performing a parallel simulation using sixteen processes.

FIG. 5 illustrates an example of matching pairwise edges based on two processes.

FIG. 6 illustrates an example of performing simulation based on two processes according to an embodiment.

FIG. 7 illustrates an example of adjusting edge forces such that magnitudes of forces of each pairwise interaction force are equal.

FIG. 8 illustrates an example of a lambda space according to an embodiment.

FIG. 9 illustrates an example of aligning pairwise edges.

FIG. 10 illustrates an example of communication of pairwise forces obtained between different processes.

FIG. 11 illustrates an example of obtaining a net force received by atom 1.

FIG. 12 illustrates a flowchart for determining, using a machine-learning model, forces of atoms in a preprocessed atomic graph that interact with respective atoms in an original atomic graph according to an embodiment.

FIG. 13 illustrates an example of matching edge pairs when a simulation space is divided into four processes.

FIG. 14 illustrates a block diagram of an MD simulation device according to an embodiment.

FIG. 15 illustrates a block diagram of an electronic device for MD simulation according to an embodiment.

### DETAILED DESCRIPTION

The following detailed structural or functional description is provided as non-limiting illustrative examples and various alterations and modifications may be made to the example embodiments without departing from the scope and spirit of the invention. Here, the example embodiments are not construed as limited to the disclosure and should be understood to include all changes, equivalents, and replacements within the idea and the technical scope of the disclosure.

Terms such as first, second, and the like, may be used herein to describe components. Each of these terminologies is not necessarily used to define an essential characteristic, order, or sequence of a corresponding component but used merely used to distinguish the corresponding component from other component(s). For example, a first component may be referred to as a second component, and similarly the second component may also be referred to as the first component.

When a description states that one component is “connected”, “coupled”, or “joined” to another component, the description may include an embodiment in which a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. Additionally, the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

As used herein, “A or B,” “at least one of A and B,” “at least one of A or B,” “A, B or C,” “at least one of A, B and C,” and “at least one of A, B, or C,” each expression may include any one of the items listed together in the corresponding one of the phrases, or any combination of two or more of the listed items.

Unless otherwise defined, all terms, including technical and scientific terms, used herein have the same meaning as commonly understood by one of ordinary skill in the art to which the present disclosure pertains.

Hereinafter, example embodiments are described in detail with reference to the accompanying drawings. When describing the embodiments with reference to the accompanying drawings, like reference numerals refer to like components, and redundant description related thereto may be omitted. For example, while each drawing may represent one or more particular embodiments of the present disclosure, drawn to scale, such that the relative lengths, thicknesses, and angles can be inferred therefrom, it is to be understood that the present invention is not necessarily limited to the relative lengths, thicknesses, and angles shown. Changes to these values may be made within the spirit and scope of the present disclosure, for example, to allow for manufacturing limitations and the like.

Molecular dynamics (MD) is a technique for studying the properties and behaviors of materials by computationally simulating and calculating the motions of atoms, molecules, or other molecular-scale particles based on interatomic or intermolecular interactions.

For example, MD simulations may be performed using simulation software such as the Large-scale Atomic/Molecular Massively Parallel Simulator (LAMMPS), which is configured to execute large-scale parallel MD calculations on distributed computing systems.

In MD simulations, a periodic boundary conditions (PBC) technique may be applied to minimize boundary effects in finite simulated systems. In MD simulations, particles are arranged within a simulation box, and a plurality of identical periodic image cells are placed around the box, such that the system effectively becomes an infinite system along one or more spatial directions without boundaries. Application of the periodic boundary conditions technique may prevent systematic errors that may occur due to finite boundary effects. For example, atoms of the system to be simulated may be arranged in a simulation box, and the surroundings may correspond to translated replicas of the system.

Embodiments of the present inventive concept provide a system and a method for performing a molecular dynamics (MD) simulation that enforces physical force symmetry in a machine-learning-based force prediction framework. For example, the system identifies and matches pairwise interaction forces predicted across space-divided atomic graphs and performs parallel simulation processes. For example, the system determines interaction force between interacting atoms of a preprocessed atomic graph including an original atomic graph and one or more mirrored replicated atomic graphs surrounding the original atomic graph. In some cases, mirrored replicated atomic graphs are generated by performing spatial decomposition of a simulation space for parallel processing. Pairwise interaction forces represent interaction relationships between pairs of atoms in a simulated system. By adjusting magnitudes of corresponding pairwise interaction forces to be equal, the system enforces Newton's third law, thereby enhances consistency, numerical stability, and energy conservation of the MD simulation while maintaining scalability under periodic boundary conditions.

In some aspects, the system improves simulation accuracy and stability by assigning consistent atom identifiers across original atomic graphs and replicated atomic graphs, mapping atomic interactions into a normalized lambda space, and determining subspace relationships for corresponding edges. For example, by aligning and communicating (using MPI) corresponding pairwise interaction forces across parallel simulation processes and adjusting the forces based on matched edge pairs, the system prevents force asymmetry introduced by spatial decomposition. Accordingly, the MD simulation method of the present disclosure improves the accuracy and efficiency of large-scale MD simulations.

Embodiments of the present disclosure improve molecular dynamics simulations by increasing physical accuracy and numerical stability in large-scale, machine-learning-based simulations executed under spatial decomposition and parallel processing. Conventional approaches independently predict interaction forces across decomposed simulation domains, which can violate Newton's third law and results in simulation instability. By contrast, the present system enforces force symmetry by matching corresponding pairwise interaction forces across replicated atomic graphs and parallel simulation processes and adjusting the magnitudes of the matched forces to be equal.

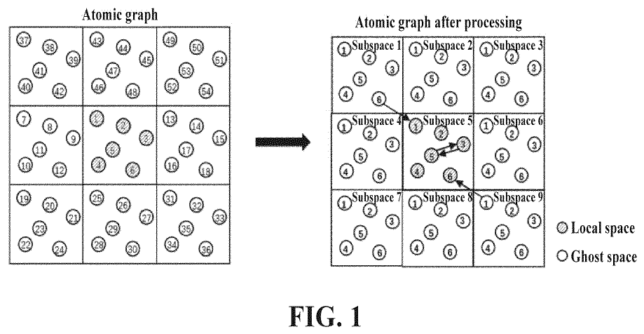

FIG. 1 illustrates an example comparing conventional technique for generation of ghost atoms by replicating local atoms in a related art MD simulation and a method for generation of ghost atoms by replicating local atoms according to an embodiment of the present disclosure.

For example, a local atom may represent an atom to be simulated (or an atom included within a target simulation domain), and a ghost atom may represent an atom generated by spatially translating the atom to be simulated for the simulation. In some cases, the ghost atom may represent an atom generated by spatially translating the atom to be simulated outside the target simulation domain for application of periodic boundary conditions. Referring to the left and right illustrations of FIG. 1, a central lattice region may correspond to a region including a local atom, and lattices surrounding the central lattice region may correspond to ghost atoms.

For example, a space corresponding to a local atom, or a space in which a local atom is positioned, may be referred to as a local space, and a space corresponding to a ghost atom, or a space in which a ghost atom is positioned, may be referred to as a ghost space or a surrounding space. In some cases, the space corresponding to a ghost atom may be referred to as a ghost space, a surrounding space, or a replicated space generated based on periodic boundary conditions.

Referring to the left illustration of FIG. 1, identifiers (IDs) (i.e., numbers or index values) assigned to atoms in different lattices may differ even when the atoms correspond to periodic replicas of an identical atomic configuration.

For convenience of description, a force between atoms may be referred to as an edge force, and a pairwise edge force may represent an interaction force acting between two atoms. An edge may be a geometric representation including a vector or a connecting line from the position of one atom to the position of another atom. For example, a vector from atom 42 to atom 1 and a vector from atom 1 to atom 42 may be referred to as a pairwise vector, a corresponding pairwise vectors, a pairwise edge, or a pairwise line, and may be denoted as f₄₂₁ and f₁₄₂, respectively. Forces on f₄₂₁ and f₁₄₂ denote a force exerted by atom 42 on atom 1 and a force exerted by atom 1 on atom 42, respectively.

For example, the force of atom 42 on atom 1 and the force of atom 1 on atom 42 may each be represented as forces associated with a pairwise edge. The force of atom 5 on atom 3 and the force of atom 3 on atom 5 may also be represented as forces associated with a pairwise edge defined between atom 5 and atom 3.

Although FIG. 1 illustrates an example of a two-dimensional arrangement of replicated atoms, the example is merely illustrative and does not necessarily limit the present disclosure. In some cases, the same principles are applicable to three-dimensional atomic configurations

For example, an electronic device may perform an MD simulation based on direct force calculation method. For example, in a direct force-based simulation method, edge features may be obtained using a graph neural network based on an atomic graph, a force on an edge may be determined using a force predictor based on the edge features, a net force applied to an atom may be determined based on forces applied by other atoms, and the position of the atom may be updated based on the net force and a simulation time step. An edge feature may represent a feature of an atom (or a characteristic associated with an atomic pair) corresponding to the edge.

A general direct force-based simulation method may enhance the prediction computational speed of a model. According to Newton's third law, magnitudes of interaction forces acting between two atoms are equal and directions of the interaction forces are opposite. However, pairwise forces predicted by a general direct force-based simulation method might not be equal, which may cause a significant loss of simulation accuracy.

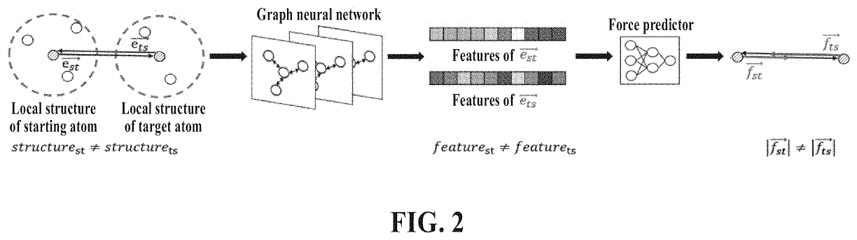

FIG. 2 illustrates an example of predicting pairwise forces.

Referring to FIG. 2, in pairwise forces such as a force vector e exerted by a starting atom on a target atom and a force vector e exerted by the target atom on the starting atom, the local structures associated with the corresponding atomic neighborhoods of the forces may differ after spatial decomposition, which may also result in different embedding features. Accordingly, the magnitude (or amplitude) of a pairwise force predicted based on these features may differ, which may conflict with Newton's third law of motion requiring equal and opposite interaction forces. Such inaccurate force prediction may reduce overall computational accuracy of a model and may cause inconsistencies including non-conservation of energy and numerical instability of the simulation.

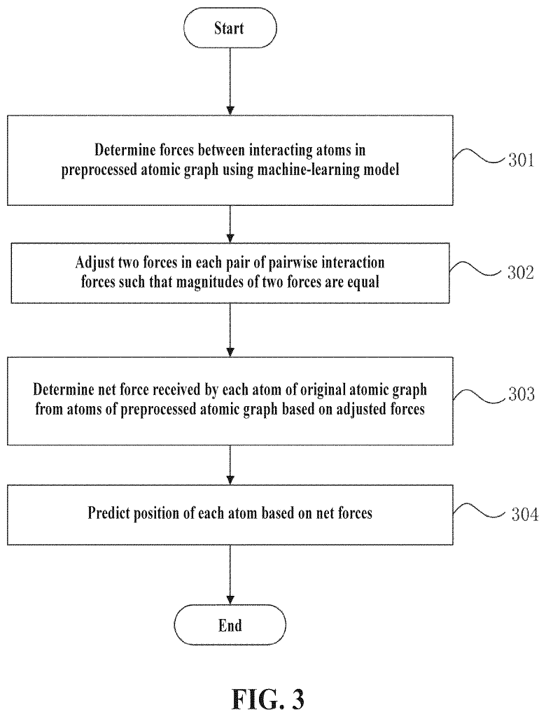

FIG. 3 illustrates a flowchart of an MD simulation method according to an embodiment.

Referring to FIG. 3, in operation 301, an electronic device (e.g., an MD simulation device) configured to perform MD simulation may determine forces between interacting atoms in a preprocessed atomic graph using a machine-learning model. In some cases, the machine-learning model may be pre-trained. The preprocessed atomic graph may include an original atomic graph and mirrored replicas of the original atomic graph surrounding the original atomic graph.

For example, the electronic device may obtain the preprocessed atomic graph by replicating the original atomic graph based on periodic boundary conditions applied along one or more spatial dimensions.

Referring again to the two-dimensional example of replicated local atoms illustrated in FIG. 1, the electronic device may obtain the preprocessed atomic graph by periodically replicating local atoms (or the original atomic graph) surrounding the local atoms (or original atoms). For example, the preprocessed atomic graph may include the original atomic graph and a plurality of periodically replicated original atomic graphs arranged around the original atomic graph.

In operation 302, the electronic device may adjust two forces (or two interaction force vectors) in each pair of pairwise interaction forces such that magnitudes of the two forces are constrained to be equal.

For example, referring to the right illustration of FIG. 1, a force exerted by atom 6 in subspace 1 on atom 1 in subspace 5 and a force exerted by atom 1 in subspace 5 on atom 6 in subspace 1 may be treated as a pairwise interaction forces associated with a single atomic interaction across subspace boundaries.

In an embodiment, the electronic device may perform an MD simulation more accurately by adjusting the forces such that magnitudes of the two forces in each pair of pairwise interaction forces are equal.

For example, the method illustrated in FIG. 3 may further include operations of assigning the same identifier to each atom of the original atomic graph and to each atom corresponding to each atom of the original atomic graph among mirrored replicas of the original atomic graph, and matching pairwise interaction forces based on the assigned identifiers of atoms of the preprocessed atomic graph.

Referring to the right illustration of FIG. 1, the electronic device may assign to a ghost atom the same identifier (or number) as an identifier of a corresponding local atom from the original atomic graph.

For example, atoms 42, 12, 24, 30, 36, 48, 18, and 54 in the surrounding space correspond to atom 6 (refer to the left illustration of FIG. 1), and the electronic device may assign the number 6 to these atoms (refer to the right illustration of FIG. 1) to indicate correspondence to a single original atom.

For example, compared to the atomic graph shown in the left illustration of FIG. 1, the preprocessed atomic graph may facilitate efficient and accurate edge matching based on atom identifiers.

For example, the pairwise interaction forces described above may include pairwise interaction forces obtained in the same simulation process and pairwise interaction forces obtained in different simulation processes. In pairwise interaction forces obtained in the same simulation process, both forces may be obtained during execution of the same simulation process. In pairwise interaction forces obtained in different simulation processes, one force may be obtained by a first simulation process, and the other force may be obtained by a second simulation process that is different from the first simulation process. In some cases, the second simulation process may be executed separately from (or simultaneously as) the first simulation process.

For example, the operation of determining forces between interacting atoms of the preprocessed atomic graph may include determining forces between interacting atoms of a first preprocessed atomic graph through a first simulation process and determining forces between interacting atoms of a second preprocessed atomic graph through a second simulation process. The first preprocessed atomic graph and the second preprocessed atomic graph may be obtained by dividing the preprocessed atomic graph, and the first simulation process and the second simulation process may be parallel simulation processes.

In some aspects, in operation 303, the electronic device (e.g., an MD simulation device) may be configured to perform MD simulation to determine net force received by each atom of original atomic graph form atoms of preprocessed atomic graph based on adjusted forces. In operation 304, the electronic device may be configured to predict position of each atom based on net forces.

In some cases, a machine learning model is a computational algorithm, model, or system designed to recognize patterns, make predictions, or perform a specific task (for example, image processing) without being explicitly programmed. According to some aspects, the machine learning model is implemented as software stored in a memory unit (e.g., the memory 1510 described with reference to FIG. 15) and executable by a processor unit (e.g., the processor 1520 described with reference to FIG. 15), as firmware, as one or more hardware circuits, or as a combination thereof.

In one aspect, machine learning model includes machine learning parameters. Machine learning parameters, also known as model parameters or weights, are variables that provide behaviors and characteristics of the machine learning model. Machine learning parameters can be learned or estimated from training data and are used to make predictions or perform tasks based on learned patterns and relationships in the data.

Machine learning parameters are adjusted during a training process to minimize a loss function or maximize a performance metric. The goal of the training process is to find optimal values for the parameters that allow the machine learning model to make accurate predictions or perform well on the given task.

For example, during the training process, an algorithm adjusts machine learning parameters to minimize an error or loss between predicted outputs and actual targets according to optimization techniques like gradient descent, stochastic gradient descent, or other optimization algorithms. Once the machine learning parameters are learned from the training data, the machine learning parameters are used to make predictions on new, unseen data.

According to some embodiments, the machine learning model includes a transformer (or a transformer model, or a transformer network), where the transformer is a type of neural network model used for natural language processing tasks. A transformer network transforms one sequence into another sequence using an encoder and a decoder. The encoder and decoder include modules that can be stacked on top of each other multiple times. The modules comprise multi-head attention and feed-forward layers. The inputs and outputs (target sentences) are first embedded into an n-dimensional space. Positional encoding of the different words (e.g., give each word/part in a sequence a relative position since the sequence depends on the order of its elements) is added to the embedded representation (n-dimensional vector) of each word. In some examples, a transformer network includes an attention mechanism, where the attention looks at an input sequence and decides at each step which other parts of the sequence are important. The attention mechanism involves a query, keys, and values denoted by Q, K, and V, respectively. Q is a matrix that contains the query (vector representation of one word in the sequence), K are the keys (vector representations of the words in the sequence) and V are the values, which are again the vector representations of the words in the sequence. For the encoder and decoder, multi-head attention modules, V consists of the same word sequence as Q. However, for the attention module that takes into account the encoder and the decoder sequences, V is different from the sequence represented by Q. In some cases, values in V are multiplied and summed with some attention-weights.

During the training process, the one or more node weights are adjusted to increase the accuracy of the result (e.g., by minimizing a loss function that corresponds in some way to the difference between the current result and the target result). The weight of an edge increases or decreases the strength of the signal transmitted between nodes. In some cases, nodes have a threshold below which a signal is not transmitted at all. In some examples, the nodes are aggregated into layers. Different layers perform different transformations on the corresponding inputs. The initial layer is known as the input layer and the last layer is known as the output layer. In some cases, signals traverse certain layers multiple times.

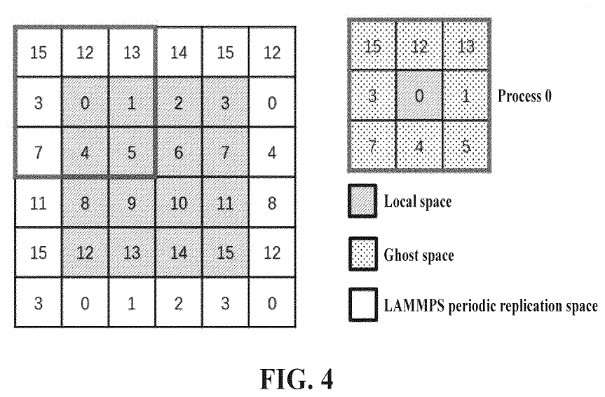

FIG. 4 illustrates an example of performing a parallel simulation using a plurality of processes, such as sixteen parallel processes.

Referring to FIG. 4, an electronic device may perform an MD simulation by dividing an original atomic graph into, for example, sixteen simulation processes. The gray boxes in FIG. 4 correspond to all atoms included in the original atomic graph, and a number in each box indicates a simulation process identifier corresponding to atoms in the box. For example, atoms in gray box 0 may be simulated through simulation process 0 to obtain forces received acting on the atoms. The atomic graph shown in the left illustration of FIG. 4 may be obtained by replicating atoms in the gray boxes to generate ghost atoms for boundary interactions. The atomic graph shown in the right illustration of FIG. 4 may represent forces applied to an intermediate atom (or target atom located in a local space) by other atoms through execution of simulation process 0 including interactions with atoms in ghost spaces. In some cases, these simulations steps may be repeated for sixteen simulation processes (e.g., simulation process 0 to simulation process 15).

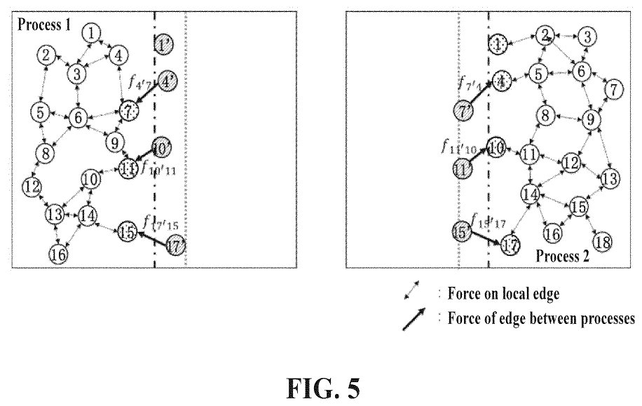

FIG. 5 illustrates an example of matching pairwise edges based on two parallel simulation processes.

Referring to FIG. 5, an electronic device may obtain forces on edges f_{4′7}, f_{10′11}, and f_{17′15} in process 1.

The electronic device may obtain the forces on the edges f_{7′4}, f_{11′10}, and f_{15′17} in process 1. As described above, edges f_{4′7} and f_{7′4}, edges f_{10′11} and f_{11′10}, and edges f_{17′15} and f_{15′7} may each form an edge pair (or a corresponding edge pair). Accordingly, forces on edges f_{4′7}, f_{10′11}, and f_{17′15} and forces on edges f_{7′4}, f_{11′10}, and f_{1t′17} may each be treated as corresponding pairwise interaction forces.

In a three-dimensional simulation space, each local space may have twenty-six spatial directions. For example, each local space may have twenty-six neighboring spatial directions corresponding to adjacent replicated regions. Accordingly, FIG. 5 may merely illustrate an example of matched edge pairs in a single spatial direction.

For example, pairwise forces obtained from different processes may be synchronized and exchanged through inter-process communication based on the Message Passing Interface (MPI) to enable force matching and adjustment across process boundaries.

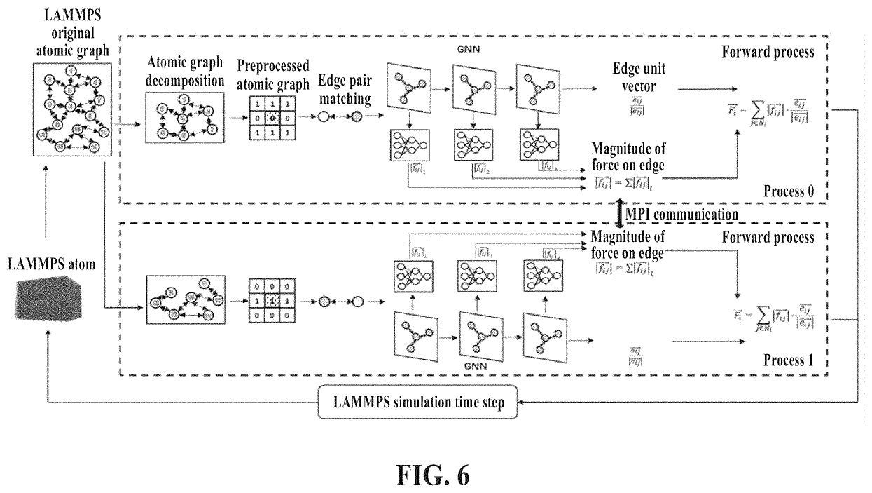

FIG. 6 illustrates an example of performing simulation based on two parallel simulation processes according to an embodiment.

Referring to FIG. 6, an electronic device may simulate forces received by atoms of a preprocessed original atomic graph through each process. For example, the electronic device may obtain forces on atomic edges of a first preprocessed atomic graph (e.g., preprocessed atomic graph 1) in process 0 and obtain forces on atomic edges of a second preprocessed atomic graph (e.g., preprocessed atomic graph 2) in process 1. The electronic device may synchronize corresponding pairwise forces obtained from different processes through MPI-based communication to enable cross-process force consistency.

For example, the electronic device may synchronize forces on edges f_{4′7}, f_{10′11}, and f_{17′15} obtained in process 0 with process 1 and synchronize forces on edges f_{7′4}, f_{10′11}, and f_{15′17} obtained in process 1 with process 0 based on matching atom identifiers and edge pairs.

For example, the above-described operation of adjusting forces may include obtaining an average value (e.g., an arithmetic average value) of the magnitudes of two forces of a pairwise interaction force and assigning the average value as the magnitude of each of the two forces.

Referring to FIG. 6, an electronic device may obtain an original atomic graph corresponding to an MD simulation domain partitioned into two parallel simulation processes. The electronic device may perform atomic graph decomposition for each simulation process to generate a corresponding preprocessed atomic graph for each process, each preprocessed atomic graph including local atoms and replicated ghost atoms generated based on periodic boundary conditions. In each simulation process, the electronic device may perform edge pair matching on the corresponding preprocessed atomic graph based on atom identifiers assigned to local atoms and corresponding ghost atoms, and may input matched edge pair information to a graph neural network (GNN). The GNN may process edge features derived from local atomic environments to generate an edge unit vector representing an interaction direction and a magnitude of force on a corresponding edge for each matched edge pair. The electronic device may provide the edge unit vector and the magnitude of force on the edge output from the GNN to a forward pass operation, and the forward pass operation may compute a force vector for each interacting atomic pair based on the edge unit vector and the magnitude of force on the edge, and may further determine a net force applied to each atom within the original atomic graph for a simulation time step.

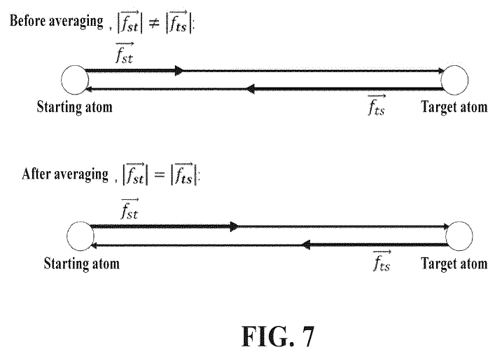

FIG. 7 illustrates an example of adjusting edge forces such that magnitudes of forces of each pairwise interaction force are equal.

Referring to FIG. 7, before the force adjustment, magnitudes of pairwise interaction forces might not be equal due to independent force prediction for each interaction direction. An electronic device may make magnitudes of pairwise interaction forces equal after adjustment by computing an average of the magnitudes of the two interaction forces and assigning the average magnitude to both interaction forces.

The above-described average-based adjustment method is merely an example and does not necessarily limit the present disclosure. In addition, other methods may be used to make magnitudes of pairwise interaction forces equal, for example, selecting a smaller magnitude force, a larger magnitude force, or selecting a magnitude based on a predefined rule or weighting scheme as the magnitude of the adjusted force.

For example, the method illustrated in FIG. 3 may further include mapping a preprocessed atomic graph to a lambda space including a plurality of subspaces, determining an identifier of a subspace corresponding to one of two atoms of each interaction force pair, determining an identifier of a subspace corresponding to the other atom of the two atoms based on the identifier of the subspace corresponding to the one atom, and determining whether adjustment of each interaction force pair is required based on a comparison of the identifiers of the subspaces corresponding to the two atoms.

As described above, a local space may represent a space corresponding to an original atomic graph or original atoms. A ghost space may represent a space corresponding to a replicated atomic graph, replicated atoms, and may also be referred to as a surrounding space. For example, a preprocessed atomic graph obtained by periodically replicating an original atomic graph surrounding the original atomic graph may include a local space and a ghost space (or a surrounding space). The local space may correspond to the original atomic graph or original atoms, and the ghost space or surrounding space may correspond to the replicated atomic graph or replicated atoms.

For example, the electronic device may map a simulation space to a lambda space based on the following equation.

$$[ Equation 1 ]$$

$$\tilde{x} = \begin{matrix}{H}^{- 1} ( x - {x}_{0} ) , & when the simulation space is triclinic ; \\ x , & when the simulation space is orthogonal .\end{matrix} \qquad ( 1 )$$

Here, \tilde{x} represents coordinates in a lambda space (e.g., defined as a cubic simulation space with a side length of 1), and the coordinates in the lambda space may be used for atom tracking and might not be used for energy and force calculations. H represents an upper-triangular 3×3 cell matrix of a triclinic space, and x represents a coordinate in Cartesian space. x₀represents a lower bound of the triclinic space.

For example, the electronic device may determine an identifier of a subspace to which a relative edge belongs based on the following equation.

$$[ Equation 2 ]$$

$$edge\_cell \_shift ( \vec{{e}_{st}} ) = ⌊ ( \tilde{{x}_{s}} - \text{?} / \text{?} ⌉ \qquad ( 2 )$$

Here, edge_cell_shift may represent a relative shift between an adjacent subspace and a local subspace. For example, a relative shift may be an integer value such as −2, −1, 0, 1, or 2, and force synchronization may be performed by retaining only edges with relative shifts of −1, 0, and 1 corresponding to immediately adjacent subspaces. \vec{eₛₜ} represents an edge from a starting (or ghost) atom s to a target (or local) atom t, and xₛ represents coordinates of the starting (or ghost) atom s. midpoint may represent coordinates of a geometric midpoint of a current process simulation space, and subbox may represent the spatial size or dimension of a local subspace.

$$[ Equation 3 ]$$

$$subspace\_ID ( \vec{{e}_{st}} ) = side\_zise \times edge\_cell \_shift ( \vec{{e}_{st}} ) [ 0 ] + edge\_size \times edge\_cell \_shift ( \vec{{e}_{st}} ) [ 1 ] + edge\_cell \_shift ( \vec{{e}_{st}} ) [ 2 ] + mid\_ID \qquad ( 3 )$$

Here, when subspace _ID(\vec{eₜₛ})=max _ID−subspace _ID(\vec{eₛₜ}), [0], [1], and [2] denote three-dimensional indices in a lambda space.

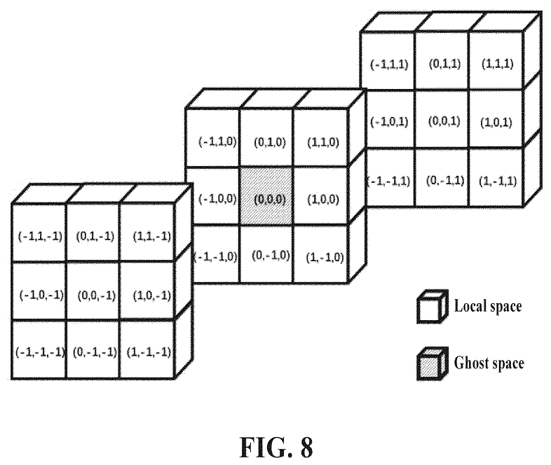

FIG. 8 illustrates an example of a lambda space according to an embodiment.

Referring to FIG. 8, box_size=3³=27, side_size=3²=9, edge_size=3, max_ID=box_size−1, and mid_ID=1/2×max_ID. Here, box_size, side_size, and edge_size may represent the size of a lambda space, the size of a side, and the size of an edge, respectively.

As described above, a subspace to which a relative edge or a pairwise edge belongs may represent a subspace in which an atom (or ghost atom) associated with the relative edge or the pairwise edge is positioned based on mapped lambda coordinates.

For example, in process 0, an identifier of a subspace to which f_{4 7}, f_{10′11}, and f_{17′15} belong may be 0. For example, when an identifier of a subspace to which 4′, 10′ and 17′ belong is 0, the identifier of a subspace to which f_{7′4}, f_{11′10}, and f_{15′17} belong in process 1 may be 26. Similarly, when an identifier of a subspace to which 4′, 10′, and 17′ belong is 1, the identifier of a subspace to which f_{7′4}, f_{11′10}, and f_{15′17} belong may be 25.

For example, an electronic device may realign edge data within each simulation process based on the obtained subspace identifiers. For example, when an opposing edge exists in another process, a corresponding adjustment of the pairwise interaction force may be needed to enforce force symmetry.

In an embodiment, the electronic device may efficiently and accurately synchronize forces on pairwise edges after determining both the simulation process and the subspace to which a relative edge belongs.

For example, the electronic device may further align forces on pairwise edges across simulation processes to generate adjusted interaction forces used for net force accumulation.

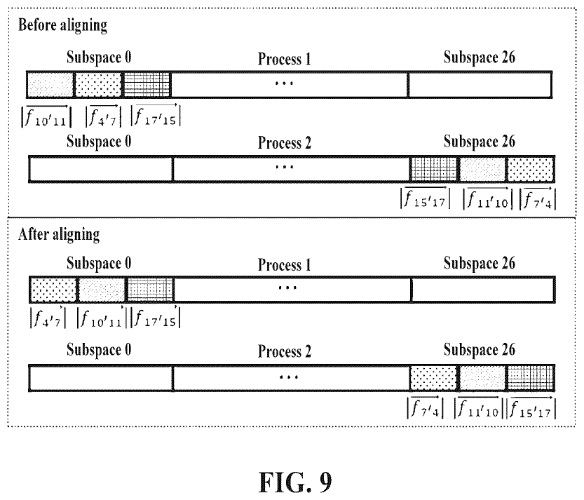

FIG. 9 illustrates an example of aligning pairwise edges.

For example, an electronic device may align edges based on the numerical values of atomic identifiers or numbers. For example, the electronic device may align edges based on numerical values of atom identifiers assigned to local atoms and corresponding ghost atoms, such that pairwise edges associated with identical atom identifier pairs are ordered consistently across processes.

Referring to FIG. 9, forces on pairwise edges may be more readily synchronized after alignment operation, thereby enabling accurate matching, adjustment, and reconciliation of interaction forces obtained from different simulation processes. For example, the electronic device may perform a pairwise edge alignment operation by sorting or reordering edge data within each simulation process based on a predetermined ordering rule using atom identifier pairs associated with each edge. For example, for each edge representing an interaction between a first atom and a second atom, the electronic device may generate an ordered atom identifier pair based on identifiers assigned to the first atom and the second atom, and may align edges across different simulation processes by matching identical ordered atom identifier pairs. For example, before the alignment, the edge might not exhibit an order. After the alignment, the edges in each subspace, and in each process, may show an order of f_{4′7}, f_{10′11}, and f_{17′15}. However, the order of the edges are not necessarily limited thereto.

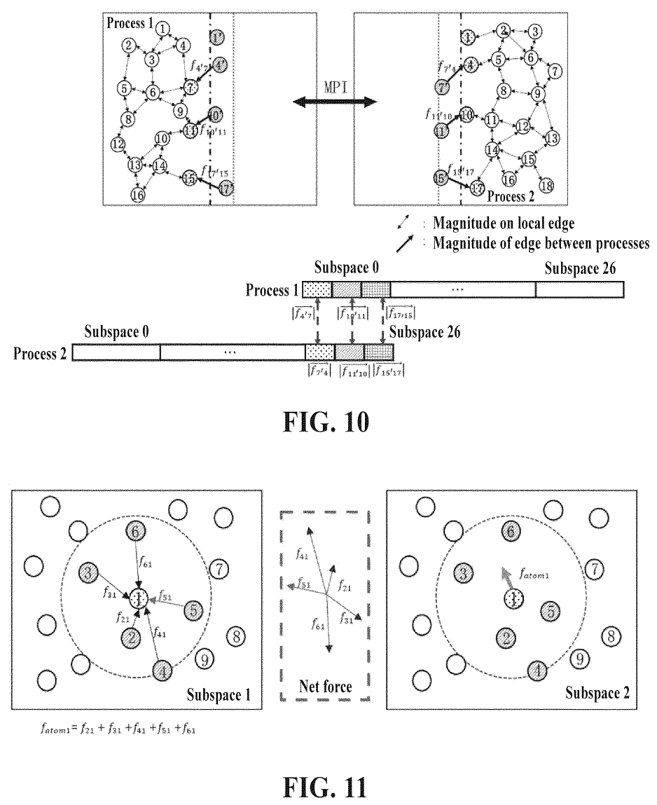

FIG. 10 illustrates an example of communication of pairwise forces obtained between different processes.

Referring to FIG. 10, an electronic device may adjust forces based on magnitudes of pairwise interaction forces for each of process 1 and process 2 through inter-process force communication, such that magnitudes of corresponding pairwise interaction forces are matched across process boundaries.

Referring back to FIG. 3, in operation 303, the electronic device may determine a net force (or a resultant net force) received by each atom of an original atomic graph from atoms of a preprocessed atomic graph based on the adjusted pairwise interaction forces.

For example, a net force received by an atom may be obtained based on the following equation.

$$[ Equation 4 ]$$

$$\vec{{F}_{i}} = {\sum }_{j ∈ {N}_{i}} \vert \vec{{f}_{ij}} \vert \cdot \frac{\vec{{e}_{J}}}{\vert \vec{{e}_{J}} \vert} \qquad ( 4 )$$

FIG. 11 illustrates an example of obtaining a net force received by atom 1.

Referring back to FIG. 3, in operation 304, an electronic device may predict an updated position of each atom based on net forces. The electronic device may determine updated coordinates of each atom based on the net forces applied to the atom and a simulation step size.

For example, a machine-learning model may include a graph neural network and a plurality of force prediction networks. A graph neural network (GNN) may include a plurality of cascaded layers. The operation of determining a force between interacting atoms of a preprocessed atomic graph may include inputting the preprocessed atomic graph to a first layer of a graph neural network, and inputting features output from a previous layer to each layer to obtain features output from each layer and determining forces between interacting atoms of the preprocessed atomic graph based on the features output from each layer using a force prediction network corresponding to each layer.

A GNN is a type of neural network designed to operate on graph-structured data. In some cases, the GNN can process various sizes of graphs having different levels of complexities. For example, the nodes of the GNN represent entities depicted in the graph-structured data, and the edges of the GNN represent relationships between the entities depicted in the graph-structured data. In some aspects, a GNN uses the graph structure to aggregate and propagate information across nodes, and captures local and global patterns within the graph-structured data.

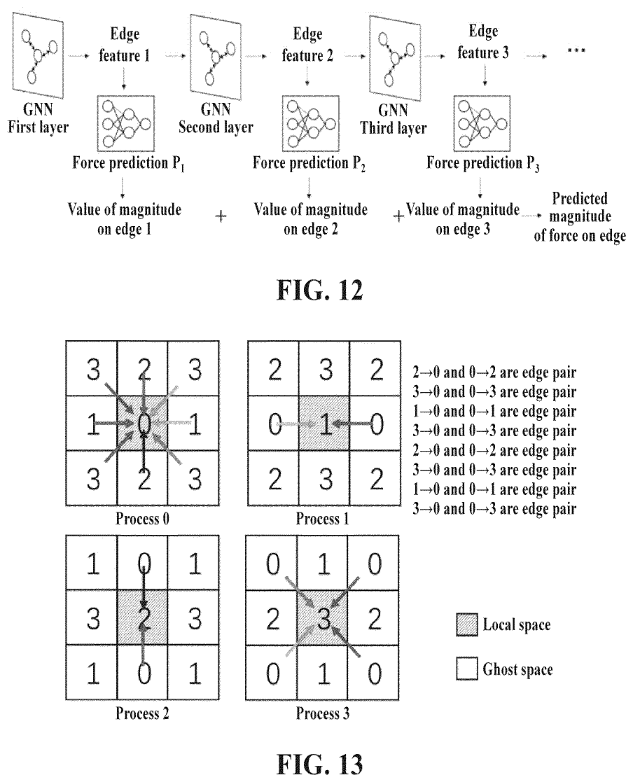

FIG. 12 illustrates a flowchart for determining, using a machine-learning model, forces of atoms in a preprocessed atomic graph that interact with respective atoms in an original atomic graph according to an embodiment.

Referring to FIG. 12, the machine-learning model may include a GNN and a plurality of force prediction networks, such as three force prediction networks. In some cases, the machine-learning model may be a pre-trained model. In some cases, the machine-learning model may be fine-tuned using a task-specific dataset corresponding to an MD simulation domain. A graph neural network may include a plurality of cascaded layers, such as three layers. In some cases, the GNN may include more than three layers.

The electronic device may input the preprocessed atomic graph to a first layer of the GNN to obtain a first edge feature (e.g., edge feature 1). The electronic device may input edge feature 1 to a second layer of the GNN to obtain a second edge feature, (e.g., edge feature 2). The electronic device may input edge feature 2 to a third layer of the GNN to obtain a third edge feature (e.g., edge feature 3). The electronic device may input the first, second, and third edge features to corresponding force predictors P1, P2, and P3, respectively, to obtain plurality of force magnitude values (e.g., the first, second, and third force values) on edges. The electronic device may obtain a final predicted force magnitude on edges based on the force magnitude values obtained from the plurality of force predictors. In contrast, a conventional system may only predict forces on edges using one edge feature.

In an embodiment, obtaining a final predicted force magnitude on edges based on force magnitude values generated from a plurality of graph neural network layers may result in higher precision or accuracy and faster convergence.

A machine-learning model, according to an embodiment, may be applied to a space-divided atomic graph generated through spatial decomposition and periodic replication.

For example, based on the training process or training configuration of the model, the electronic device may obtain a final predicted force magnitude on edges by directly summing output from the force prediction networks or using a weighted summation. For example, summation weights may be obtained through model training and stored as model parameters.

As described above, after pairwise edge matching edges, a simulation process to which a pairwise edge belongs and a subspace to which the pairwise edge belongs may be identified, and based on the identified process and subspace, a corresponding force on a relative edge may be obtained and used to adjust the force on the relative edge to enforce force symmetry.

FIG. 13 illustrates an example of matching edge pairs in an MD simulation environment when a simulation space is divided into four parallel simulation processes.

Referring to FIG. 13, for example, a force exerted by an atom positioned in a ghost space and calculated in process 0 on an atom in a local space, and forces exerted by atoms in ghost spaces calculated in each of the other three processes on atoms in the local space, may be identified as corresponding pairwise interaction forces and matched as edge pairs.

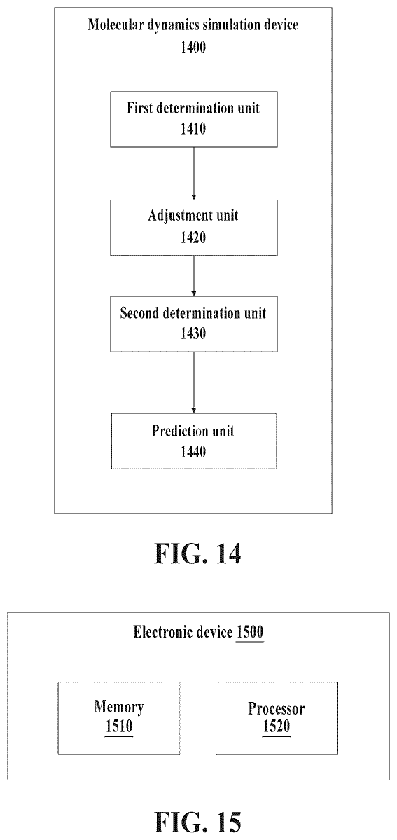

The MD simulation method has been described above with reference to FIGS. 1 to 13. Hereinafter, a device for MD simulation, according to an embodiment, are described with reference to FIG. 14.

FIG. 14 illustrates a block diagram of an MD simulation device according to an embodiment.

Referring to FIG. 14, an MD simulation device 1400 may include a first determination unit 1410, an adjustment unit 1420, a second determination unit 1430, and a prediction unit 1440.

The MD simulation device 1400 may further include one or more additional components, and at least one of the components included in the MD simulation device 1400 may be implemented as a separate hardware module or integrated with another component.

For example, the first determination unit 1410 may determine forces between interacting atoms of a preprocessed atomic graph using a machine-learning model. The preprocessed atomic graph may include an original atomic graph and a plurality of mirrored replicas of the original atomic graph surrounding the original atomic graph to support periodic boundary condition processing.

For example, the adjustment unit 1420 may adjust two interaction force vectors in each pair of pairwise interaction forces such that magnitudes of the two interaction force vectors are equal.

For example, the second determination unit 1430 may determine a net force received by each atom of the original atomic graph from atoms of the preprocessed atomic graph based on the adjusted pairwise interaction forces.

For example, the prediction unit 1440 may predict an updated position of each atom based on net forces and a simulation time step.

For example, the pairwise interaction forces may include pairwise interaction forces obtained in the same simulation process and pairwise interaction forces obtained in different simulation processes. For example, both forces in a pair of pairwise interaction forces obtained in the same simulation process may be obtained from the same (or a single) simulation process. Among pairwise interaction forces obtained from different simulation processes, one force may be obtained from a first simulation process, and the other force may be obtained from a second simulation process different from the first simulation process.

For example, the first determination unit 1410 may determine forces between interacting atoms of a first preprocessed atomic graph through a first simulation process and determine forces between interacting atoms of a second preprocessed atomic graph through a second simulation process. For example, the first preprocessed atomic graph and the second preprocessed atomic graph may be obtained by spatially dividing the preprocessed atomic graph. The first simulation process and the second simulation process may be executed as parallel simulation processes.

For example, the MD simulation device 1400 may further include a matching module. The matching module may assign the same identifier to each atom in the original atomic graph and to each atom corresponding to each atom in the original atomic graph among the replicated original atomic graphs. The matching module may match pairwise interaction forces based on identifiers of atoms in the preprocessed atomic graph.

For example, the MD simulation device 1400 may further include a third determination unit. The third determination unit may map the preprocessed atomic graph to a lambda space, where the lambda space may include a plurality of subspaces. The third determination unit may determine an identifier of a subspace corresponding to one of two atoms in each interaction force pair. The third determination unit may determine an identifier of a subspace corresponding to the other atom based on the identifier of the subspace corresponding to the one atom. The third determination unit may determine whether adjustment of each interaction force pair is required based on a comparison of the identifier of the subspace corresponding to one atom and the identifier of the subspace corresponding to the other atom.

For example, the adjustment unit 1420 may obtain an average value of the magnitudes of the two forces of a pairwise interaction force and use the average value as the magnitude of each of the two forces.

For example, a machine-learning model may include a GNN and a plurality of force prediction networks. A GNN may include a plurality of cascaded layers. The first determination unit 1410 may input features output from a previous layer of each layer of the GNN to a subsequent layer and obtain features output from each respective layer. For example, an input to a first layer of the GNN may be the preprocessed atomic graph. The first determination unit 1410 may determine forces between interacting atoms of the preprocessed atomic graph based on features output from each layer, using a corresponding force prediction network associated with each layer.

FIG. 15 illustrates a block diagram of an electronic device for MD simulation according to an embodiment.

According to an embodiment, an electronic device 1500 may include, but is not necessarily limited to, a desktop computer, a laptop computer, a tablet computer, a server system, or a supercomputer.

A processor 1520 may execute, for example, software (e.g., one or more programs or instructions) to control at least one other component (e.g., a hardware or software component) of the electronic device 1500 connected to the processor 1520 and may perform various data processing or computation. In an embodiment, as at least a part of data processing or computation, the processor 1520 may store a command or data received from another component in a volatile memory 1510, process the command or the data stored in the volatile memory 1510, and store resulting data in a non-volatile memory. The processor 1520 may perform at least one of the operations described with reference to FIGS. 1 to 13.

The memory 1510 may store various pieces of data used by at least one component (e.g., the processor 1520) of the electronic device 1500. The various pieces of data may include, for example, software (e.g., one or more programs) and input data or output data for a command related thereto. The memory 1510 may include volatile memory, non-volatile memory, or a combination thereof. The memory 1510 may store information for performing operations of the MD simulation method described above with reference to FIGS. 1 to 13. For example, the memory 1510 may store a program in which operations of the MD simulation method described above with reference to FIGS. 1 to 13 are implemented.

The electronic device 1500 may include the processor 1520 and the memory 1510. The processor 1520 may perform operations (e.g., operations of the MD simulation method described with reference to FIGS. 1 to 13) of the above-described units (e.g., the first determination unit 1410, the adjustment unit 1420, the second determination unit 1430, the prediction unit 1440, the matching module, and the third determination unit of FIG. 14). The memory 1510 may store one or more instructions. The one or more instructions stored in the memory 1510, when executed by the processor 1520, may cause the electronic device 1500 to perform the MD simulation method. For example, the one or more instructions stored in the memory 1510, when executed by the processor 1520, may cause the electronic device 1500 to perform at least one of determining a force between interacting atoms of a preprocessed atomic graph including an original atomic graph and a mirrored atomic graph surrounding the original atomic graph using a machine-learning model, adjusting two forces of each pair of pairwise interaction forces such that magnitudes of the two forces are equal, determining a net force received by each atom of the original atomic graph from atoms of the preprocessed atomic graph based on the adjusted forces, and predicting the position of each atom based on the net force.

The example embodiments described herein may be implemented using a hardware component, a software component, or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor or any other processing device capable of responding to and executing instructions. The processing device may run an operating system (OS) and one or more software applications that run on the OS. The processing unit also may access, store, manipulate, process, and generate data in response to execution of the software. For purpose of simplicity, the description of a processing unit is used as singular. However, one skilled in the art will appreciate that a processing unit may include a plurality of processing elements and a plurality of types of processing elements. For example, the processing unit may include a plurality of processors, or a single processor and a single controller. In addition, different processing configurations are possible, including parallel processing configurations.

The software may include a computer program, a piece of code, one or more instructions, or a combination thereof, to individually or collectively instruct or configure the processing device to operate as desired. Software and/or data may be stored in any type of machine, component, physical or virtual equipment, or computer storage medium or device capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The method according to the embodiment may be implemented in the form of program instructions that can be executed through various computer means and recorded on a computer-readable medium. The media may also include, alone or in combination with the program instructions, data files, data structures, and the like. Examples of non-transitory computer-readable media include magnetic media such as hard disks, floppy disks, and magnetic tape; optical media such as CD-ROM discs, DVDs, and/or Blue-ray discs; magneto-optical media such as optical discs; or hardware devices that are specially configured to store and perform program instructions, such as read-only memory (ROM), random-access memory (RAM), flash memory (e.g., USB flash drives, memory cards, memory sticks, etc.), and the like. Examples of program instructions include both machine code, such as generated by a compiler, and files containing higher-level code that may be executed by the computer using an interpreter.

The above-described devices may be configured to act as one or more software modules in order to perform the operations of the above-described examples, or vice versa.

As described above, although the embodiments have been described with reference to the drawings, a person skilled in the art may apply various technical modifications and variations based thereon without departing from the scope of the disclosure. For example, suitable results may be achieved if the described techniques are performed in a different order and/or if components in a described system, architecture, device, or circuit are combined in a different manner and/or replaced or supplemented by other components or functionally equivalent components.

What is claimed is:

1. An electronic device comprising:
at least one processor; and
a memory storing instructions,
wherein the instructions, when executed individually or collectively by the at least one processor, cause the electronic device to:
determine, using a machine-learning model, an interaction force between interacting atoms of a preprocessed atomic graph including an original atomic graph and a mirrored replicated atomic graph surrounding the original atomic graph, wherein the mirrored replicated atomic graph is generated by performing spatial decomposition of a simulation space in the original atomic graph;
adjust magnitudes of two forces of each pair of pairwise interaction forces of the interacting atoms;
determine a net force applied to each of the interacting atoms in the original atomic graph based on the adjusted magnitudes; and
generate an updated position of each atom based on the net force.

2. The electronic device of claim 1, wherein the pairwise interaction forces include pairwise interaction forces obtained in a same simulation process and pairwise interaction forces obtained in a different simulation process,
wherein among the pairwise interaction forces obtained in the same simulation process, forces are obtained in the same simulation process, and among the pairwise interaction forces obtained in the different simulation process, a first force is obtained from a first simulation process and a second force is obtained from a second simulation process different from the first simulation process.

3. The electronic device of claim 2, wherein the instructions, when executed individually or collectively by the at least one processor, cause the electronic device to:
obtain a first preprocessed atomic graph and a second preprocessed atomic graph by spatially dividing the preprocessed atomic graph;
determine forces between interacting atoms of the first preprocessed atomic graph through the first simulation process; and
determine forces between interacting atoms of the second preprocessed atomic graph through the second simulation process,
wherein the first simulation process and the second simulation process are executed as parallel simulation processes.

4. The electronic device of claim 3, wherein the instructions, when executed individually or collectively by the at least one processor, cause the electronic device to:
assign an identifier to each atom of the original atomic graph;
assign the identifier to each atom in the mirrored replicated atomic graph corresponding to each atom of the original atomic graph; and
match pairwise interaction forces based on the identifier of atoms of the preprocessed atomic graph.

5. The electronic device of claim 4, wherein the instructions, when executed individually or collectively by the at least one processor, cause the electronic device to:
synchronize the matched pairwise interaction forces through inter-process communication using Message Passing Interface (MPI) between the first simulation process and the second simulation process.

6. The electronic device of claim 4, wherein the instructions, when executed individually or collectively by the at least one processor, cause the electronic device to:
align pairwise edges corresponding to the pairwise interaction forces based on the identifier assigned to atoms associated with the pairwise edges; and
synchronize the matched pairwise interaction forces between the first simulation process and the second simulation process based on the aligned pairwise edges.

7. The electronic device of claim 1, wherein the instructions, when executed individually or collectively by the at least one processor, cause the electronic device to:
map the preprocessed atomic graph to a lambda space including a plurality of subspaces;
determine a first identifier of a subspace corresponding to a first atom of two atoms of each interaction force pair;
determine a second identifier of a subspace corresponding to a second atom of the two atoms based on first identifier; and
determine whether to adjust each interaction force pair based on a comparison of the first identifier and the second identifier.

8. The electronic device of claim 1, wherein the instructions, when executed individually or collectively by the at least one processor, cause the electronic device to:
compute an average value of magnitudes of two forces of pairwise interaction forces.

9. The electronic device of claim 1, wherein the machine-learning model includes a graph neural network and a plurality of force prediction networks,
wherein the graph neural network includes a plurality of cascaded layers, and
the instructions, when executed individually or collectively by the at least one processor, cause the electronic device to:
input the preprocessed atomic graph to a first layer of the graph neural network to generate an output feature; and
determine forces between interacting atoms of the preprocessed atomic graph based on the output feature.

10. A method performed by an electronic device, the method comprising:
determining, using a machine-learning model, an interaction force between interacting atoms of a preprocessed atomic graph including an original atomic graph and a mirrored replicated atomic graph surrounding the original atomic graph, wherein the mirrored replicated atomic graph is generated by performing spatial decomposition of a simulation space in the original atomic graph;
adjusting magnitudes of two forces of each pair of pairwise interaction forces of the interacting atoms;
determining a net force applied to each of the interacting atoms in the original atomic graph based on the adjusted magnitudes; and
generating an updated position of each atom based on the net force.

11. The method of claim 10, wherein the pairwise interaction forces include at least one of pairwise interaction forces obtained in a same simulation process and pairwise interaction forces obtained in a different simulation process,
among the pairwise interaction forces obtained in the same simulation process, forces are obtained in the same simulation process, and
among the pairwise interaction forces obtained in the different simulation process, a first force is obtained from a first simulation process and a second force is obtained from a second simulation process different from the first simulation process.

12. The method of claim 11, wherein the determining of the force between the interacting atoms of the preprocessed atomic graph comprises:
obtaining a first preprocessed atomic graph and a second preprocessed atomic graph by spatially dividing the preprocessed atomic graph;
determining forces between interacting atoms of the first preprocessed atomic graph through the first simulation process; and
determining forces between interacting atoms of the second preprocessed atomic graph through the second simulation process,
wherein the first simulation process and the second simulation process are executed as parallel simulation processes.

13. The method of claim 12, further comprising:
assigning an identifier to each atom of the original atomic graph;
assign the identifier to each atom in the mirrored replicated atomic graph corresponding to each atom of the original atomic graph; and
matching pairwise interaction forces based on the identifier of atoms in the preprocessed atomic graph.

14. The method of claim 13, further comprising:
synchronizing the matched pairwise interaction forces between the first simulation process and the second simulation process.

15. The method of claim 14, wherein the synchronizing of the matched pairwise interaction forces comprises:
aligning pairwise edges corresponding to the pairwise interaction forces based on the identifier assigned to atoms associated with the pairwise edges; and
synchronizing the matched pairwise interaction forces between the first simulation process and the second simulation process based on the aligned pairwise edges.

16. The method of claim 10, further comprising:
mapping the preprocessed atomic graph to a lambda space including a plurality of subspaces;
determining a first identifier of a subspace corresponding to a first atom of two atoms of each interaction force pair;
determining a second identifier of a subspace corresponding to a second atom of the two atoms based on the first identifier; and
determining whether to adjust each interaction force pair based on a comparison of the first identifier and the second identifier.

17. The method of claim 10, wherein the adjusting of the two forces comprises:
computing an average value of magnitudes of two forces of pairwise interaction forces.

18. The method of claim 10, wherein the machine-learning model includes a graph neural network and a plurality of force prediction networks,
wherein the graph neural network includes a plurality of cascaded layers, and
wherein determining the force between the interacting atoms of the preprocessed atomic graph comprises:
inputting the preprocessed atomic graph to a first layer of the graph neural network to generate an output feature; and
determining forces between the interacting atoms of the preprocessed atomic graph based on the output feature.

19. A computer-readable storage medium storing executable program instructions, wherein the executable program instructions, when executed by a processor, cause the processor to:
determine, using a machine-learning model, an force between interacting atoms of a preprocessed atomic graph including an original atomic graph and a mirrored replicated atomic graph surrounding the original atomic graph, wherein the mirrored replicated atomic graph is generated by performing spatial decomposition of a simulation space in the original atomic graph;
adjust magnitudes of two forces of each pair of pairwise interaction forces of the interacting atoms;
determine a net force applied to each of the interacting atoms in the original atomic graph based on the adjusted magnitudes; and
generate an updated position of each atom based on the net force.

20. The computer-readable storage medium of claim 19, wherein the executable program instructions, when executed by a processor, further cause the processor to:
assigning an identifier to each atom of the original atomic graph;
assign the identifier to each atom coin the mirrored replicated atomic graph corresponding to each atom of the original atomic graph; and
matching pairwise interaction forces based on the identifier of atoms in the preprocessed atomic graph.
